(19) United States
(12) Patent Application Publication
(10) Pub. No.: US 2002/0077291 A1
(43) Pub. Date: Jun. 20, 2002

(54) Method of treatment of tumors using transforming growth factor-alpha

(76) Inventors: Alan Upshall, Kenmore, WA (US); Daniel R. Twardzik, Banbridge Island, WA (US); Andrzej Sledziewski, Shoreline, WA (US)

Correspondence Address: GARY CARY WARE & FRIENDENRICH LLP, 4365 EXECUTIVE DRIVE, SUITE 1600, SAN DIEGO, CA, 92121-2189, US

(21) Appl. No.: 09/961,227

(22) Filed: Sep. 20, 2001

Related U.S. Application Data
(60) Provisional application No. 60/235,152, filed on Sep. 22, 2000.

(51) Int. Cl. A61K 38/18; A61K 31/7048; A61K 31/704; A61K 31/522; A61K 31/664

(52) U.S. Cl. 514/12; 514/8; 514/34; 514/105; 514/263.31; 514/27; 514/575

(57) ABSTRACT

The present invention provides pharmaceutical compositions and methods for treating cell proliferative disorders, such as tumors, in a subject, utilizing TGF-α or functional fragments thereof. Optionally, a chemotherapeutic agent is administered in combination with TGF-α or fragments thereof.

### FIELD OF THE INVENTION

The present invention relates generally to growth factors and more specifically to the use of transforming growth factor alpha (TGF-α) for inhibition or suppression of tumor cell growth, in the presence or absence of chemotherapeutic agents.

### BACKGROUND OF THE INVENTION

Cancers are the leading cause of death in animals and humans. The exact cause of cancer is not known, but links between certain activities such as smoking or exposure to carcinogens and certain inherited factors, and the incidence of certain types of cancers and tumors has been shown by a number of researchers.

Many types of chemotherapeutic agents have been shown to be effective against cancers and tumor cells, but not all types of cancers and tumors respond to these agents. Unfortunately, many of these agents are toxic and also destroy normal cells. The exact mechanism for the action of these chemotherapeutic agents are not always known.

Despite advances in the field of cancer treatment, the leading therapies to date are surgery, radiation and chemotherapy. Chemotherapeutic approaches are often used for cancers that are metastasized or ones that are particularly aggressive. Such cytocidal or cytostatic agents work best on cancers whose cells are rapidly dividing. To date, hormones, in particular estrogen, progesterone and testosterone, and some antibiotics produced by a variety of microbes, alkylating agents, and anti-metabolites form the bulk of therapies available to oncologists. Ideally cytotoxic agents that have specificity for cancer and tumor cells while not affecting normal cells would be extremely desirable. Unfortunately, none have been found and instead agents which target especially rapidly dividing cells (both tumor and normal) have been used.

Clearly, the development or identification of drugs or agents that would target tumor cells due to some unique specificity for them would be a breakthrough. Alternatively, drugs or agents that are cytotoxic to tumor cells while exerting mild effects on normal cells would be desirable. It is believed that the some agents, when used in conjunction with chemotherapeutic agents can both reduce and suppress the growth of cancers, tumors and leukemia, and reduce the toxicity of the chemotherapeutic agent. Therefore, it is an object of this invention to provide a pharmaceutical composition that is effective in suppressing and inhibiting the growth of tumors and cancers in mammals with mild or no effects on normal cells or with a protective effect on healthy cells.

### SUMMARY OF THE INVENTION

The present invention is based on the seminal discovery that transforming growth factor-alpha (TGF-α) inhibits or suppresses tumor cell growth in animals. Although many chemotherapeutic agents are effective for suppressing tumor cell growth, they tend to be toxic for normal cells as well. The present invention shows that not only does TGF-α provide protection from toxicity to chemotherapeutic agents, but TGF-α by itself appears to be effective in suppressing tumor cell growth in vivo.

In a first embodiment, the invention provides a pharmaceutical composition for treating cell proliferative disorders comprising a pharmaceutically acceptable carrier, a therapeutically effective amount of TGF-α, TGF-α-related polypeptide, or a functional fragment of TGF-α or TGF-α-related polypeptide. In one aspect, the pharmaceutical composition contains a therapeutically effective amount of a chemotherapeutic agent, such as alkylating agents, DNA strand-breaking agents, intercalating topoisomerase II inhibitors, nonintercalating topoisomerase II inhibitors, DNA minor groove binders, antimetabolites, tubulin-binding agents that when bound to tubulin prevent formation of microtubules, hormones, asparaginase and hydroxyurea. In a preferred composition, the chemotherapeutic agent is cisplatin.

In another embodiment, the invention provides a method of treating a cell proliferative disorder in a mammal including administering to a subject in need thereof, a therapeutically effective amount of TGF-α, TGF-α-related polypeptide, or functional fragment thereof, thereby treating the disorder. In one aspect, the method includes co-administration of a chemotherapeutic agent, either prior to, simultaneously with, or substantially following TGF-α administration. Exemplary cell proliferative disorders include, but are not limited to, cancers and tumors such as lung cancer, pancreatic cancer, colon cancer, myeloid leukemia, melanoma, glioma, thyroid follicular cancer, bladder carcinoma, myelodysplastic syndrome, breast cancer, low grade astrocytoma, astrocytoma, glioblastoma, medulloblastoma, renal cancer, prostate cancer, endometrial cancer and neuroblastoma.

The invention also includes a method of treatment of a cell proliferative disorder in a subject in need thereof. The method includes introducing into cells of a host subject, an expression vector comprising a polynucleotide sequence encoding TGF-α or a biologically functional fragment thereof, in operable linkage with a promoter.

### BRIEF DESCRIPTION OF THE FIGURES

FIG. 1 is a graph showing the effect of TGF-α on cisplatin efficacy in human epidermal cancer model (A431) (see Example 1) as exemplified by mean tumor volume. Treatment courses included: PBS, TGF-α, cisplatin, and cisplatin plus TGF-α. Treatment was carried out over 26 days. Arrows indicate day of treatment with cisplatinum (also referred to as cisplatin) and XXX indicates day of treatment with gfa50 (gfa50, as used herein refers to TGF-α).

FIG. 2 is a graph showing the effect of TGF-α on cisplatin toxicity in human epidermal cancer model (A431) (see Example 1) as exemplified by mean body weight. Treatment courses included: PBS, TGF-α, cisplatin, and cisplatin plus TGF-α. Treatment was carried out over 26 days. Arrows indicate day of treatment with cisplatinum (cisplatin) and XXX indicates day of treatment with gfa50 (gfa50, as used herein refers to TGF-α).

FIG. 3 is a graph showing the effect of TGF-α on cisplatin efficacy in human epidermal cancer model (A431) (see Example 1) as exemplified by mean tumor volume. Treatment courses included: PBS, TGF-α, cisplatin, and cisplatin plus TGF-α. Treatment was carried out over 26 days. Arrows indicate day of treatment with cisplatinum (cisplatin) and XXX indicates day of treatment with gfa50 (gfa50, as used herein refers to TGF-α).

FIG. 4 is a graph showing the effect of TGF-α on cisplatin toxicity in human epidermal cancer model (A431) (see Example 1) as exemplified by mean body weight. Treatment courses included: PBS, TGF-α, cisplatin, and cisplatin plus TGF-α. Treatment was carried out over 26 days. Arrows indicate day of treatment with cisplatinum (cisplatin) and XXX indicates day of treatment with gfa50 (gfa50, as used herein refers to TGF-α).

### DETAILED DESCRIPTION OF THE INVENTION

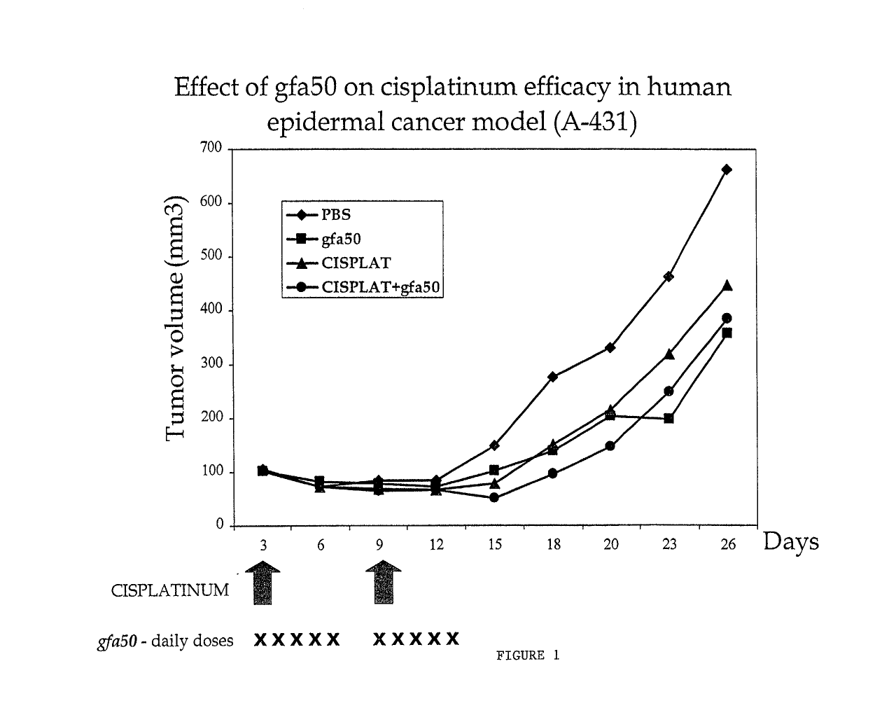

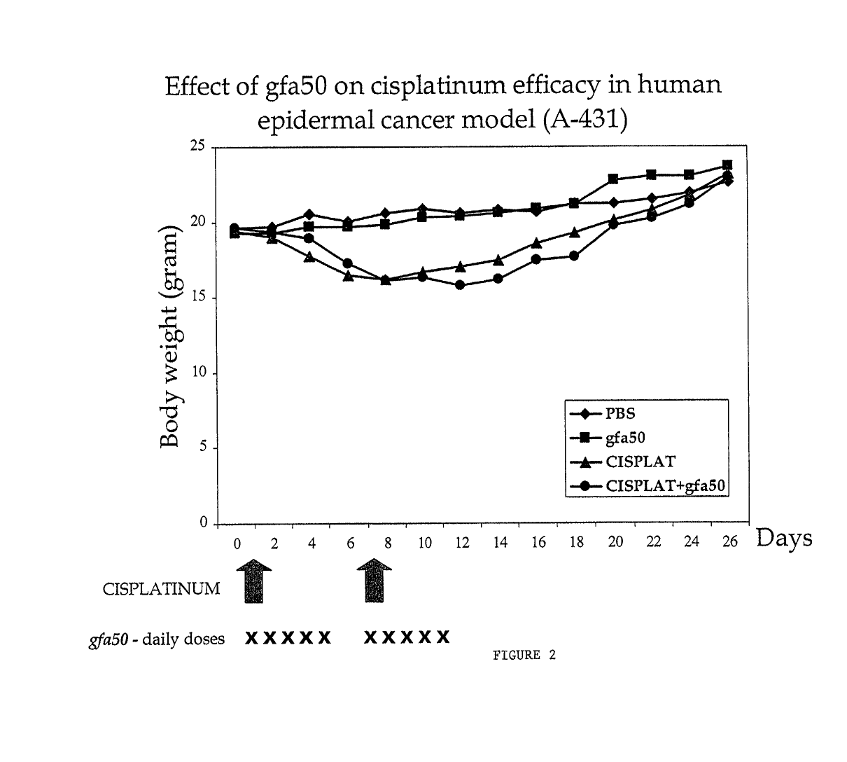

In accordance with the present invention, there are provided pharmaceutical compositions and methods of use for the treatment of tumors in a subject. The invention is based on the discovery that TGF-α is effective for suppressing or inhibiting tumor cell growth. In a particular illustrative model, the inventors have shown that TGF-α alone was as effective as cisplatin for suppressing tumor cell growth in a human epidermal cancer model (A431 cells; see Example 1 and FIGS. 1 and 2). A431 cells have a high density of epidermal growth factor (EGF) receptors on their surface, which may be related to TGF-α's action on these tumors.

TGF-α is a member of the epidermal growth factor (EGF) family and interacts with one or more receptors in the EGF-family of receptors. TGF-α stimulates the receptors' endogenous tyrosine kinase activity which results in activating various cellular functions, such as stimulating a mitogenic or migration response in a wide variety of cell types. TGF-α and EGF mRNAs reach their highest levels and relative abundance (compared to total RNA) in the early postnatal period and decrease thereafter, suggesting a role in embryonic development. From a histological perspective, TGF-α is found in numerous cell types and tissues throughout the body. The active form of TGF-α is derived from a larger 30-35 kD precursor and contains 50 amino acids. Human TGF-α shares only a 30% structural homology with the 53-amino acid form of EGF, but includes conservation and spacing of all six cysteine residues. TGF-α is highly conserved among species. For example, the rat and human polypeptides share about 90% homology compared to a 70% homology as between the rat and human EGF polypeptide. TGF-α shares cysteine disulfide bond structures with a family of TGF-α related proteins including vaccinia growth factor, amphiregulin precursor, betacellulin precursor, betacellulin, heparin binding EGF-like growth factor, epiregulin (rodents), HUS 19878, myxomavirus growth factor (MGF), Shope fibroma virus growth factor (SFGF), and schwannoma derived growth factor. Such TGF-α related polypeptides are also useful in the compositions and methods of the invention.

TGF-α is an acid and heat stable polypeptide of about 5.6 kDa molecular weight. It is synthesized as a larger 30-35 kDa molecular weight glycosylated and membrane-bound precursor protein wherein the soluble 5.6 kDa active form is released following specific cleavage by an elastase-like protease. TGF-α binds with high affinity in the nanomolar range and induces autophosphorylation of one or more members of EGF receptor family (e.g., ErbB1 through 4 or receptors that bind a neuregulin ligand) to transduce subsequent signal pathways with the EGF receptors. TGF-α is 50 amino acids in length and has three disulfide bonds to form its tertiary configuration. TGF-α is stored in precursor form in alpha granules of some secretory cells.

Human TGF-α is a polypeptide of 50 amino acids (see U.S. Pat. No. 5,240,912, Todaro et al., herein incorporated by reference). The human or rat TGF-α polypeptide can be divided roughly into three loop regions corresponding roughly (starting at the N terminus) to amino acids 1-21, to amino acids 16-32, and to amino acids 33-50. As discussed more fully below, the invention provides “functional fragments of TGF-α” that retain TGF-α biological activity. “Functional fragment” as used herein means a TGF-α “peptide that is a fragment or a modified fragment of a full length TGF-α” polypeptide or related polypeptide so long as the fragment retains some TGF-α related biological activity (e.g., interacts with an EGF family receptor, stimulates proliferation, migration, and/or differentiation of stem cells, exerts a cytoprotective effect, or is useful for treating or preventing cachexia). Other biological activities associated with the polypeptides of the invention include, for example, mitogenic effects on stem cells and their more differentiated progeny of various tissues (e.g., epithelial stem cells, hematopoietic stem cells, neural stem cells, liver stem cells, keratinocyte stem cells, and pancreatic derived stem cells).

The invention provides a TGF-α, TGF-α related polypeptide, or a functional fragment of TGF-α, TGF-α related polypeptide having TGF-α activity. The functional fragments have an altered (compared to the naturally occurring molecule) sequence, for example, the N-terminal region of TGF-α (defined as the first seven N-terminal amino acids before the first loop region) and an altered “tail” region (defined as the last seven amino acids at the C-terminus after the third loop region) can be modified, truncated or deleted as described more fully herein. The alterations to the fifty amino acid sequence of human TGF-α (SEQ ID NO: 1) caused by deletion of some or all of the seven amino acids at the N-terminal region resulted in a polypeptides having about 90% of the biological activity of the TGF-α having a sequence as set forth in SEQ ID NO: 1. In addition, substitution of D amino acids for natural L amino acids in the N-terminal region results in retention of TGF-α biological activity and an increase in plasma half life of the polypeptide after intravenous administration. Truncation of the N-terminus by 6 residues leaves a Lys residue at amino acid position 7 which provides for two free amino groups. This provides a site for forming a PEG (polyethylene glycol) “PEGalated TGFα mimetic” to be synthesized and further provides for improved pharmacokinetic benefits, including resistance to proteolytic enzyme breakdown.

The polypeptides of the invention are intended to include substantially purified naturally occurring proteins, as well as those which are recombinantly or synthetically synthesized. In addition, a TGF-α or related polypeptide can occur in at least two different conformations wherein both conformations have the same or substantially the same amino acid sequence but have different three dimensional structures so long as they have a biological activity related to TGF-α. Polypeptide or protein fragments of TGF-α retaining a cytoprotective effect are also encompassed by the invention. Fragments can have the same or substantially the same amino acid sequence as the naturally occurring protein. A polypeptide or peptide having substantially the same sequence means that an amino acid sequence is largely, but not entirely, the same, but retains a functional activity of the sequence to which it is related. In general polypeptides of the present invention include peptides, or full length protein, that contain substitutions, deletions, or insertions into the protein backbone, that would still have an approximately 50%-70% homology to the original protein over the corresponding portion. A yet greater degree of departure from homology is allowed if like-amino acids, i.e. conservative amino acid substitutions, do not count as a change in the sequence. A TGF-α polypeptide fragment of the invention retains a biological activity associated with TGF-α as described above.

Homology to TGF-α polypeptide can be measured using standard sequence analysis software (e.g., Sequence Analysis Software Package of the Genetics Computer Group, University of Wisconsin Biotechnology Center, 1710 University Avenue, Madison, Wis. 53705; also see Ausubel, et al., supra). Such procedures and algorithms include, for example, a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Anduced Multi-sequence Alignment), SAGA (Sequence Alignment by Genetic Algorithm) and WHAT-AF.

A polypeptide substantially related but for a conservative variation is encompassed by the invention. A conservative variation denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine. The term “conservative variation” also includes the use of a substituted amino acid in place of an unsubstituted parent amino acid provided that antibodies raised to the substituted polypeptide also immunoreact with the unsubstituted polypeptide.

Modifications and substitutions are not limited to replacement of amino acids. For a variety of purposes, such as increased stability, solubility, or configuration concerns, one skilled in the art will recognize the need to introduce, (by deletion, replacement, or addition) other modifications. Examples of such other modifications include incorporation of rare amino acids, dextra (D)-amino acids, glycosylation sites, cytosine for specific disulfide bridge formation. The modified peptides can be chemically synthesized, or the isolated gene can be site-directed mutagenized, or a synthetic gene can be synthesized and expressed in bacteria, yeast, baculovirus, tissue culture and so on.

Solid-phase chemical peptide synthesis methods can also be used to synthesize the polypeptide or fragments of the invention. Such methods have been known in the art since the early 1960's (Merrifield, R. B., J. Am. Chem. Soc., 85, 2149-2154 (1963) (See also Stewart, J. M. and Young, J. D., Solid Phase Peptide Synthesis, 2 ed., Pierce Chemical Co., Rockford, Ill., pp. 11-12)) and have recently been employed in commercially available laboratory peptide design and synthesis kits (Cambridge Research Biochemicals). Such commercially available laboratory kits have generally utilized the teachings of H. M. Geysen et al, Proc. Natl. Acad. Sci., USA, 81, 3998 (1984) and provide for synthesizing peptides upon the tips of a multitude of “rods” or “pins” all of which are connected to a single plate. When such a system is utilized, a plate of rods or pins is inverted and inserted into a second plate of corresponding wells or reservoirs, which contain solutions for attaching or anchoring an appropriate amino acid to the pin's or rod's tips. By repeating such a process step, i.e., inverting and inserting the rod and pin tips into appropriate solutions, amino acids are built into desired peptides. In addition, a number of available FMOC peptide synthesis systems are available. For example, assembly of a polypeptide or fragment can be carried out on a solid support using an Applied Biosystems, Inc. Model 431A automated peptide synthesizer. For example, if the peptide is from formula I or formula II (see below), a preferred means for synthesizing peptides of 10-18 amino acids in length is by direct peptide synthesis generally starting with the N-terminal amino acid and adding amino acids in the C terminal direction. TGFα has been made using recombinant techniques and is available as a laboratory reagent commercially. The bifunctional compounds of formula III are best synthesized with each loop peptide moiety synthesized and then added to the heterocyclic nitrogen atom using standard heterocyclic addition synthesis.

Generally, the terms “treating”, “treatment” and the like are used herein to mean affecting a subject, tissue or cell to obtain a desired pharmacologic and/or physiologic effect. The effect may be prophylactic in terms of completely or partially preventing a disease or disorder or sign or symptom thereof, and/or may be therapeutic in terms of a partial or complete cure for a disorder or disease and/or adverse effect attributable to the disorder or disease. “Treating” as used herein covers any treatment of, or prevention of, or inhibition of a disorder or disease in a subject. The subject can be an invertebrate, a vertebrate, or a mammal, and particularly a human.

The invention includes various pharmaceutical compositions useful for delivery or administration of the polypeptides, peptides and mimetics of the invention. In one embodiment, the pharmaceutical compositions are useful in treating tumors. The pharmaceutical compositions according to the invention are prepared by bringing a polypeptide or peptide derivative of TGF-α, a TGF-α mimetic into a form suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

The pharmaceutical compositions are preferably prepared and administered in dose units. Solid dose units are tablets, capsules and suppositories and including, for example, alginate based pH dependent release gel caps. For treatment of a subject, depending on activity of the compound, manner of administration, nature and severity of the disorder, age and body weight of the subject, different daily doses are necessary. Under certain circumstances, however, higher or lower daily doses may be appropriate. The administration of the daily dose can be carried out both by single administration in the form of an individual dose unit or by several smaller dose units and also by multiple administration of subdivided doses at specific intervals.

In one embodiment, the pharmaceutical composition further comprises a therapeutically effective amount of a chemotherapeutic agent. Such chemotherapeutic agents include alkylating agents, DNA strand-breaking agents, intercalating topoisomerase II inhibitors, nonintercalating topoisomerase II inhibitors, DNA minor groove binders, antimetabolites, tubulin-binding agents that when bound to tubulin prevent formation of microtubules, hormones, asparaginase and hydroxyurea.

The pharmaceutical composition according to claim 4 wherein said chemotherapeutic agent is selected from the group consisting of asparaginase, hydroxyurea, cisplatin, cyclophosphamide, altretamine, bleomycin, dactinomycin, doxorubicin, etopo side, teniposide, and plicamycin.

The pharmaceutical composition according to claim 4 wherein said chemotherapeutic agent is selected from the group consisting of Methotrexate, Fluorouracil, Fluorodeoxyuridine, CB3717, Azacitidine, Cytarabine, Floxuridine, Mercaptopurine, 6-Thioguanine, Fludarabine, Pentostatin, Cyctrabine, and Fludarabine.

The pharmaceutical compositions according to the invention may be administered locally or systemically in a therapeutically effective dose. Amounts effective for this use will, of course, depend on the severity of the cell proliferative disease and general state of the subject. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249: 1527, (1990); Gilman et al. (eds.) (1990), each of which is herein incorporated by reference.

In embodiments where TGF-alpha polypeptide is administered to a subject, the dosage range is about 0.1 ug/kg to 100 mg/kg; more preferably from about 1 ug/kg to 100 mg/kg and most preferably from about 1 ug/kg to 50 mg/kg.

In one embodiment, the invention provides a pharmaceutical composition useful for administering a TGF-α polypeptide or functional fragment, or a nucleic acid encoding a TGF-α polypeptide or functional fragment, to a subject in need of such treatment. “Administering” the pharmaceutical composition of the invention may be accomplished by any means known to the skilled artisan. Preferably a “subject” refers to a mammal, most preferably a human. “Therapeutically-effective” as used herein, refers to that amount of TGF-α that is of sufficient quantity to alleviate a symptom of the disease or to arrest the growth of the tumor as compared to an untreated tumor. The effective amount results in biologically active stable TGF-α for a period of time such that one or more symptoms of the disease/disorder is alleviated.

The pharmaceutical composition of the invention can be administered by standard methods including, but not limited to parenterally, enterically, by injection, rapid infusion, nasopharyngeal absorption, dermal absorption, rectally, intracranially, by aerosol or particle delivery, inhalation, intrathecally and orally. Pharmaceutically acceptable carrier preparations for parenteral administration include sterile or aqueous or non-aqueous solutions, suspensions, and emulsions. Examples of non-aqueous solvents are propylene glycol, polyethylene glycol, vegetable oils such as olive oil, and injectable organic esters such as ethyl oleate. Carriers for occlusive dressings can be used to increase skin permeability and enhance antigen absorption. Liquid dosage forms for oral administration may generally comprise a liposome solution containing the liquid dosage form. Suitable solid or liquid pharmaceutical preparation forms are, for example, granules, powders, tablets, coated tablets, (micro)capsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners and elixirs containing inert diluents commonly used in the art, such as purified water. Where the disease or disorder is a gastrointestinal disorder oral formulations or suppository formulations are preferred.

Sterile injectable solutions can be prepared by incorporating the active agent (TGF-α) in the required amount (e.g., about 1 u g to about 100 mg/kg) in an appropriate solvent and then sterilizing, such as by sterile filtration. Further, powders can be prepared by standard techniques such as freeze drying or vacuum drying.

In another embodiment, the active agent is prepared with a biodegradable carrier for sustained release characteristics for either sustained release in the GI tract or for target organ implantation with long term active agent release characteristics to the intended site of activity. Biodegradable polymers include, for example, ethylene vinyl acetate, polyanhydrides, polyglycolic acids, polylactic acids, collagen, polyorthoesters, and poly acetic acid. Liposomal formulation can also be used.

In one embodiment, TGF-α is transferred to tumors by gene therapy. The gene encoding TGF-α or a biologically active fragment thereof is transferred to tumor cells or carrier host cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those tumor cells are then delivered to a subject.

In this embodiment, the desired gene is introduced into a tumor cell prior to administration in vivo of the resulting recombinant cell. Such introduction can be carried out by any method known in the art, including but not limited to transfection, electroporation, microinjection, infection with a viral or bacteriophage vector containing the gene sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, Meth. Enzymol. 217:599-618, 1993; Cohen et al., Meth. Enzymol. 217:618-644, 1993; Cline, Pharmac. Ther. 29:69-92, 1985) and may be used in accordance with the present invention, provided that the necessary developmental and physiological functions of the recipient cells are not disrupted. The technique should provide for the stable transfer of the gene to the cell, so that the gene is expressible by the cell and preferably heritable and expressible by its cell progeny.

One common method of practicing gene therapy is by making use of retroviral vectors (see Miller et al., Meth. Enzymol. 217:581-599, 1993). A retroviral vector is a retrovirus that has been modified to incorporate a preselected gene in order to effect the expression of that gene. It has been found that many of the naturally occurring DNA sequences of retroviruses are dispensable in retroviral vectors. Only a small subset of the naturally occurring DNA sequences of retroviruses is necessary. In general, a retroviral vector must contain all of the cis-acting sequences necessary for the packaging and integration of the viral genome. These cis-acting sequences include: a) a long terminal repeat (LTR), or portions thereof, at each end of the vector; b) primer binding sites for negative and positive strand DNA synthesis; and c) a packaging signal, necessary for the incorporation of genomic RNA into virions. The gene to be used in gene therapy is cloned into the vector, which facilitates delivery of the gene into a cell by infection or delivery of the vector into the cell.

Adenoviruses and HIV-1 based lentiviral vectors are also of use in gene therapy. Adenoviruses are especially attractive vehicles for delivering genes to respiratory precursor cells. Adenoviruses can also be used to deliver genes to precursor cells from the liver, the central nervous system, endothelium, and muscle. Adenoviruses have the advantage of being capable of infecting non-dividing cells. Kozarsky and Wilson, Current Opinion in Genetics and Development 3:499-503, 1993, present a review of adenovirus-based gene therapy. Other instances of the use of adenoviruses in gene therapy can be found in Rosenfeld et al., Science 252:43 1434, 1991; Rosenfeld et al., Cell 68:143-155, 1992; and Mastrangeli et al., J. Clin. Invest. 91:225-234, 1993.

In a specific embodiment, the desired gene recombinantly expressed in the cell to be introduced for purposes of gene therapy comprises an inducible promoter operably linked to the coding region, such that expression of the recombinant gene is controllable by controlling the presence or absence of the appropriate inducer of transcription.

The isolation of tumor cells for use in the present invention can be carried out by any of numerous methods commonly known to those skilled in the art. For example, one common method for isolating tumor cells includes resection of the tumor.

The method of treating proliferative diseases, according to this invention, includes a method for treating (inhibiting) the abnormal growth of cells, including transformed cells, in a patient in need of such treatment (e.g., a mammal such as a human), by administering, prior to, concurrently or sequentially, an effective amount of TGF-α and an effective amount of a chemotherapeutic agent and/or radiation. Abnormal growth of cells means cell growth independent of normal regulatory mechanisms (e.g., loss of contact inhibition), including the abnormal growth of: (1) tumor cells (tumors) expressing an activated oncogene; (2) tumor cells in which an oncogene is activated as a result of oncogenic mutation in another gene; and (3) benign and malignant cells of other proliferative diseases.

In some embodiments, the methods of the present invention include methods for treating or inhibiting tumor growth in a patient in need of such treatment (e.g., a mammal such as a human) by administering, concurrently or sequentially, (1) an effective amount of TGF-α and (2) an effective amount of an antineoplastic agent and/or radiation therapy. Examples of tumors which may be treated include, but are not limited to, epithelial cancers, e.g., prostate cancer, lung cancer (e.g., lung adenocarcinoma), pancreatic cancers (e.g., pancreatic carcinoma such as, for example, exocrine pancreatic carcinoma), breast cancers, colon cancers (e.g., colorectal carcinomas, such as, for example, colon adenocarcinoma and colon adenoma), ovarian cancer, and bladder carcinoma. Other cancers that can be treated include melanoma, myeloid leukemias (for example, acute myelogenous leukemia), sarcomas, thyroid follicular cancer, and myelodysplastic syndrome.

Generally the term “growth” as used herein, is used to mean advancing development or proliferation. Growth also includes metastases, such that as a mass of cells grows, the cells are dispersed and may migrate to a secondary location. As such, any enlargement, amplification, spreading, or expansion of cells is growth as used herein.

As used herein, “antineoplastic agent” is a chemotherapeutic agent effective against cancer. The term “concurrently” is (1) simultaneously in time, or (2) at different times during the course of a common treatment schedule. Also, “sequentially” is (1) administration of one component of the method ((a) TGF-α, or (b) antineoplastic or chemotherapeutic agent and/or radiation therapy) followed by administration of the other component; after adminsitration of one component, the second component can be administered substantially immediately after the first component, or the second component can be administered after an effective time period after the first component; the effective time period is the amount of time given for realization of maximum benefit from the administration of the first component.

Classes of compounds that can be used as the chemotherapeutic agent (antineoplastic agent) include: alkylating agents, antimetabolites, natural products and their derivatives, hormones and steroids (including synthetic analogs), and synthetics. Examples of compounds within these classes are given below.

Alkylating agents (including nitrogen mustards, ethylenimine derivatives, alkyl sulfonates, nitrosoureas and triazenes): Uracil mustard, Chlormethine, Cyclophosphamide (Cytoxan.RTM.), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide.

Antimetabolites (including folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Natural products and their derivatives (including vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, paclitaxel (paclitaxel is commercially available as Taxol.RTM. and is described in more detail below in the subsection entitled “Microtubule Affecting Agents”), Mithramycin, Deoxycoformycin, Mitomycin-C, L-Asparaginase, Interferons (especially IFN-a), Etoposide, and Teniposide.

Hormones and steroids (including synthetic analogs): 17.alpha.-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Zoladex.

Synthetics (including inorganic complexes such as platinum coordination complexes): Cisplatin, Carboplatin, Hydroxyurea, Amsacrine, Procarbazine, Mitotane, Mitoxantrone, Levamisole, and Hexamethylmelamine.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA); the disclosure of which is incorporated herein by reference thereto.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the terms “safe and effective” or “therapeutically effective” amount refers to the quantity of a component which is sufficient to yield a desired therapeutic response without undue adverse side effects (such as toxicity, irritation, or allergic response) commensurate with a reasonable benefit/risk ratio when used in the manner of this invention. The specific “safe and effective amount” will, obviously, vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, a “pharmaceutical addition salts” is salt of the chemotherapeutic agent with an organic or inorganic acid. These preferred acid addition salts are chlorides, bromides, sulfates, nitrates, phosphates, sulfonates, formates, tartrates, maleates, malates, citrates, benzoates, salicylates, ascorbates, and the like.

As used herein, a “pharmaceutical carrier” is a pharmaceutically acceptable solvent, suspending agent or vehicle for delivering the anti-cancer agent to the animal or human. The carrier may be liquid or solid or liposomes and is selected with the planned manner of administration in mind.

As used herein, “cancer” refers to all types of cancers or neoplasm or malignant tumors and all types of cancers including leukemia that are found in mammals.

As used herein “chemotherapeutic agents” includes DNA-Anteractive agents, antimetabolites, tubulin-Anteractive agents, hormonal agents and others, such as asparaginase or hydroxyurea.

The chemotherapeutic agents are generally grouped as DNA-Anteractive agents, antimetabolites, tubulin-Anteractive agents, hormonal agents and others such as asparaginase or hydroxyurea. Each of the groups of chemotherapeutic agents can be further divided by type of activity or compound. The chemotherapeutic agents used in combination with TGF-α of this invention include members of all of these groups. For a detailed discussion of the chemotherapeutic agents and their method of administration, see Dorr, et al, Cancer Chemotherapy Handbook, 2d edition, pages 15-34, Appleton & Lange (Connecticut, 1994) herein incorporated by reference.

DNA-Anteractive agents include the alkylating agents, e.g. cisplatin, cyclophosphamide, altretamine; the DNA strand-breakage agents, such as bleomycin; the intercalating topoisomerase II inhibitors, e.g., dactinomycin and doxorubicin); the nonintercalating topoisomerase II inhibitors such as, etoposide and teniposide; and the DNA minor groove binder plicamycin.

The alkylating agents form covalent chemical adducts with cellular DNA, RNA, and protein molecules and with smaller amino acids, glutathione and similar chemicals. Generally, these alkylating agents react with a nucleophilic atom in a cellular constituent, such as an amino, carboxyl, phosphate, sulfhydryl group in nucleic acids, proteins, amino acids, or glutathione. The mechanism and the role of these alkylating agents in cancer therapy is not well understood. Typical alkylating agents include:

Nitrogen mustards, such as chlorambucil, cyclophosphamide, isofamide, mechlorethamine, melphalan, uracil mustard; aziridine such as thiotepa; methanesulphonate esters such as busulfan; nitroso ureas, such as carmustine, lomustine, streptozocin; platinum complexes, such as cisplatin, carboplatin; bioreductive alkylator, such as mitomycin, and procarbazine, dacarbazine and altretamine; DNA strand breaking agents include bleomycin. DNA topoisomerase II inhibitors include intercalators, such as amsacrine, dactinomycin, daunorubicin, doxorubicin, idarubicin, and mitoxantrone; nonintercalators, such as etoposide and teniposide. The DNA minor groove binder is plicamycin.

The antimetabolites interfere with the production of nucleic acids by one or the other of two major mechanisms. Some of the drugs inhibit production of the deoxyribonucleoside triphosphates that are the immediate precursors for DNA synthesis, thus inhibiting DNA replication. Some of the compounds are sufficiently like purines or pyrimidines to be able to substitute for them in the anabolic nucleotide pathways. These analogs can then be substituted into the DNA and RNA instead of their normal counterparts. The antimetabolites useful herein include folate antagonists such as methotrexate and trimetrexate; pyrimidine antagonists, such as fluorouracil, fluorodeoxyuridine, CB3717, azacitidine, cytarabine, and floxuridine; purine antagonists include mercaptopurine, 6-thioguanine, fludarabine, pentostatin; sugar modified analogs include cyctrabine, fludarabine; ribonucleotide reductase inhibitors include hydroxyurea. Tubulin interactive agents act by binding to specific sites on tubulin, a protein that polymerizes to form cellular microtubules. Microtubules are critical cell structure units. When the interactive agents bind on the protein, the cell can not form microtubules tubulin interactive agents include vincristine and vinblastine, both alkaloids and paclitaxel. Hormonal agents are also useful in the treatment of cancers and tumors. They are used in hormonally susceptible tumors and are usually derived from natural sources and include estrogens, conjugated estrogens and ethinyl estradiol and diethylstilbesterol, chlortrianisen and idenestrol; progestins such as hydroxyprogesterone caproate, medroxyprogesterone, and megestrol; androgens such as testosterone, testosterone propionate; fluoxymesterone, methyltestosterone. Adrenal corticosteroids are derived from natural adrenal cortisol or hydrocortisone. They are used because of their anti inflammatory benefits as well as the ability of some to inhibit mitotic divisions and to halt DNA synthesis. These compounds include, prednisone, dexamethasone, methylprednisolone, and prednisolone. Leutinizing hormone releasing hormone agents or gonadotropin-releasing hormone antagonists are used primarily the treatment of prostate cancer. These include leuprolide acetate and goserelin acetate. They prevent the biosynthesis of steroids in the testes. Antihormonal antigens include antiestrogenic agents such as tamosifen, antiandrogen agents such as flutamide; and antiadrenal agents such as mitotane and aminoglutethimide. Hydroxyurea appears to act primarily through inhibition of the enzyme ribonucleotide reductase. Asparagenase is an enzyme which converts asparagine to nonfunctional aspartic acid and thus blocks protein synthesis in the tumor.

The present invention also provides gene therapy for the treatment of cell proliferative disorders. Such therapy would achieve its therapeutic effect by introduction of the TGF-alpha polynucleotide or fragments thereof encoding biologically active molecules of TGF-alpha into cells having the proliferative disorder. Delivery of such polynucleotides can be achieved using a recombinant expression vector such as a chimeric virus or a colloidal dispersion system. Other preferred methods for therapeutic delivery of sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. By inserting a TGF-alpha sequence of interest into the viral vector, along with another gene which encodes the ligand for a receptor on a specific target cell, for example, the vector is now target specific. Retroviral vectors can be made target specific by attaching, for example, a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome or attached to a viral envelope to allow target specific delivery of the retroviral vector containing the polynucleotide.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packaging mechanism to recognize an RNA transcript for encapsulation. Helper cell lines which have deletions of the packaging signal include, but are not limited to [symbol] 2, PA317 and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Alternatively, NIH 3T3 or other tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for polynucleotides is a colloidal dispersion system. Colloidal dispersion systems include macromolecule complexes, nanocapsules, microspheres, beads, and lipid-based systems including oil-An-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 u m can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. in order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Manning, et al., Biotechniques, 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidyiglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the lipo some to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

The invention will now be described in greater detail by reference to the following non-limiting examples.

### EXAMPLES

### EXAMPLE 1

### PROTOCOL FOR PRECLINICAL STUDY TESTING EFFECT OF TGF-α ON

### CISPLATIN EFFICACY USING THE METAMOUSE™ MODEL OF TUMOR GROWTH

Using a nude mouse model and human epidermal cancer cell tumor (A-431), having 10⁷ or greater EGF receptors, the inventors investigated whether TGF-alpha would promote tumor growth. In a parallel experiment, the tumor was treated with a chemotherapeutic agent, cisplatinum. The TGF-alpha effect on the cisplatinum efficacy was observed. In a further parallel experiment, the inventors investigated whether TGF-alpha would directly influence the survival of the tumor. Phase 1 of the protocol called for treatments with cisplatinum at day 3 at 10 mg/kg and treatment with TGF-alpha, 5 injections each treatment=days 3 through 7, at 50 ug/kg. Five mice in three of the four groups were sacrificed at this time. Phase 2 of the protocol called for injection with cisplatinum at 10 mg/kg on day 9 and treatment with TGFa at 50 micrograms/kg on days 9-13. Phase 3 began on day 20. Each group of 10 mice was split into: 1) A no further treatment group, (Phase 3A); and 2) a group that received one more injection of cisplatinum at 10 mg/kg on day 20 and TGF-alpha on days 20-24 at 50 ug/kg) (phase 3B).

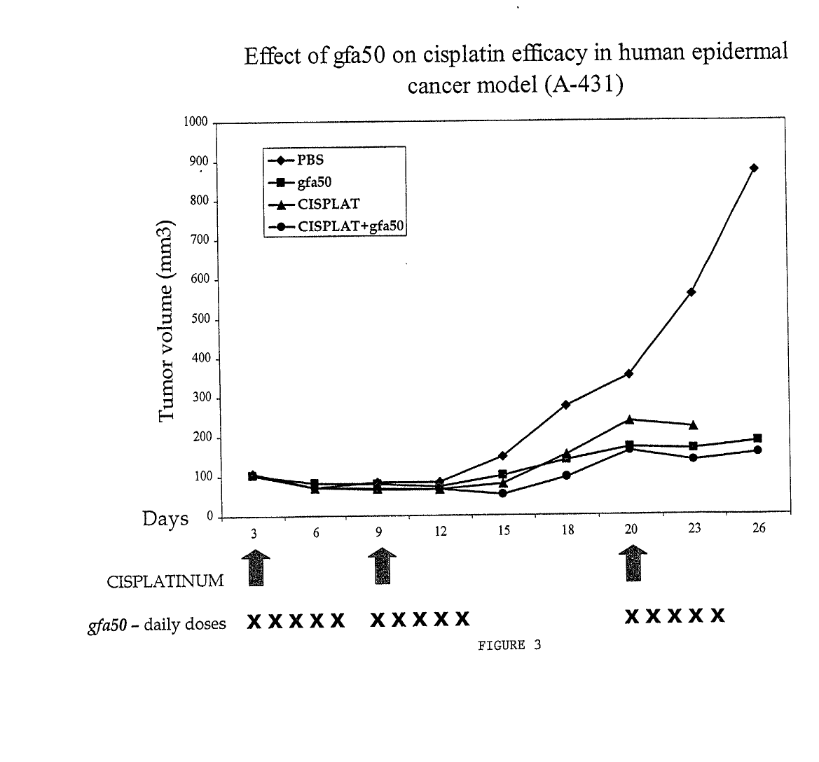

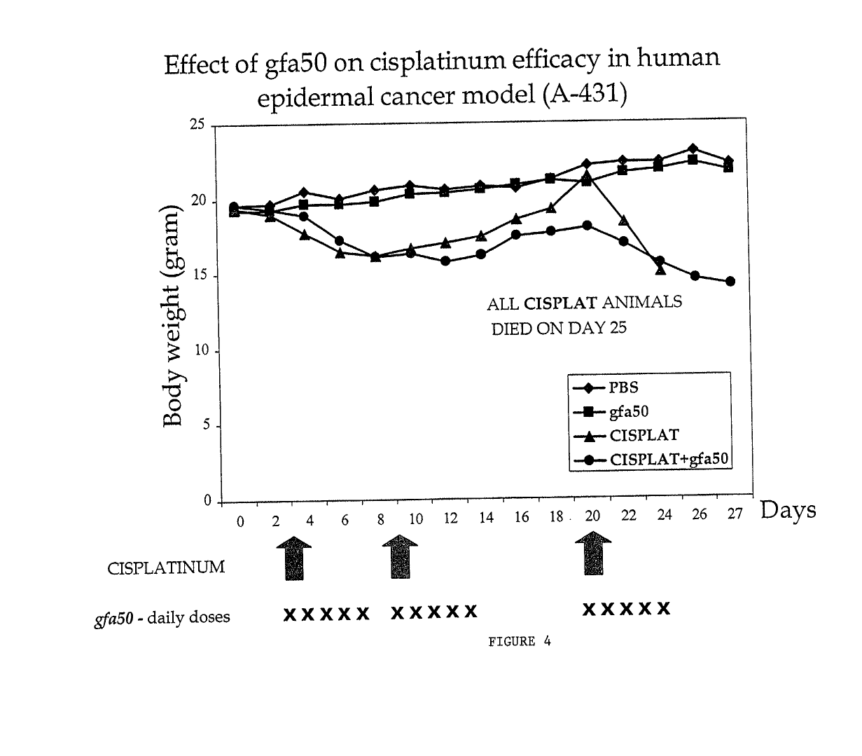

The results seen in FIG. 1 and 2 refer to phases 1, 2 and phase 3A courses of treatment (days 3 and 9 for cisplatinum and days 3-7 and 9-13 for TGF-alpha) and in FIGS. 3 and 4 phases 1,2 and 3B (days 3, 9 as above and day 20 for cisplatinum and days 3-7, 9-13 and 20-24 for TGF-alpha).

The results show that while cisplatinum alone is more effective than the control for treatment of tumors, cisplatinum tends to be toxic (see FIG. 4). TGF-alpha, either alone, or in combination with cisplatinum, inhibited tumor growth (FIGS. 1 and 3) and protected against toxicity of cisplatinum (FIGS. 2 and 4). In addition, treatment with TGF-alpha alone, while inhibiting tumor growth to the same degree as cisplatinum treatment (FIG. 1 and 3), had no effect on body weight. In contrast, cisplatin treated mice had dramatic body weight loss at the third cisplatinum treatment followed by death (see FIG. 4). Finally, TGF-alpha protected against the death caused by the third treatment of cisplatinum (FIG. 4).

The study consisted of four groups of nude mice:

1. Group A—control (15 animals); this group was implanted with tumor and treated only with placebo (according to TGF-injection schedule).

2. Group B—TGF-controls (10 animals); this group was implanted with tumor and received only TGF-injection (according to TGF-injection schedule).

3. Group C—cisplatin treated (15 animals): this group was implanted with tumor and treated with cisplatin (according to cisplatin schedule); this group will also receive placebo (according to TGF-injection schedule).

4. Group D—cisplatin and TGF-treated (15 animals); this group was implanted with tumor and was treated with cisplatin and TGF-(according to cisplatin and TGF-injection schedules respectively).

PHASE 1 :On day 1 all animals were orthotopically implanted with tumor in their breast fat flaps (2-3 tumor tissue specimens per animal). On day 3 in the morning the tumor was measured in all animals. Then animals from group B and D received the first injection of TGF-alpha, animals from group A and C received placebo. Six hours later animals from group C and D received cisplatin injection.

TGF-alpha was injected at a concentration of 1 microgram per mouse (assuming mouse weighs 20 gram) or 50 microgram per kg.

Cisplatin was injected in concentration of 200 microgram per mouse (assuming mouse weighs 20 gram) or 10 milligram per kg.

Placebo composition—regular PBS containing no Mg++ and no Ca++ ions.

On day 4, day 5, day 6 and day 7 animals from group B and D received additional TGF-alpha injections spaced 24 hours from the first administration; at the same time, animals from group A and C received placebo.

On day 6 the tumor load in all animals was measured again.

On day 8, five animals were randomly selected from group A, C and D for sacrifice. Their tumor load was measured again, their blood drawn and their intestines, kidneys and spleen removed and preserved. The spleen weight was determined at the time of collection.

Phase 2. On day 9 animals from groups B and D received TGF-alpha injection at 50 micrograms per mouse and 6 hours later animals from groups C and D received cisplatinum injection at 50 mg/kg. On day 10, day 11, day 12 and day 13 animals from group B and D received additional TGF-alpha injections spaced 24 hours from the 9th day administration; animals from group A and C receive placebo at the same time.

On day 12 the tumor load in all animals was measured again. On day 15 all study animals were sacrificed. their tumor load was measured again, their blood drawn and their intestines, kidneys and spleen removed and preserved. The spleen weight was determined at the time of collection.

Phase 3. On day 20 in the morning the tumor load was measured in all remaining animals according to anticancer procedure. Each group of 10 (groups A, B, C, D) mice were divided into two subgroups: for phase 3A, 5 animals in each group were maintained with no further treatments). For phase 3B, 5 animals from original groups B and D received another injection of TGF-alpha and animals from original group A and C received placebo. 6 hours later animals from original group C and D received a second cisplatin injection. On day 21, 22, 23 and 24 the five animals from original groups B and D received additional TGF-alpha injections spaced 24 hours from the 20th day of administration; the five selected animals from original group a and c received placebo at the same time. The experiment was terminated on day 26.

Summary of assays for all 3 phases;

body weight—daily for all animals

tumor load (GFP assay)—days 3, 6, 9, 12 and 15 for all animals; plus for animals sacrificed at the end of the phase one—day 8

blood chemistry—all animals, baseline on day 1 (probably using capillaries) plus for animals sacrificed on day 8 and day 26 respectively

spleen weights of all animals after sacrifice

histopathology—collect tumor tissue, intestines, kidneys and spleen from all animals sacrificed on day 8 plus the same organs from five animals from each experimental group on day 26. The small intestine processing: jejunum in two pieces; longitudinal about 1 cm, cross in 0.5 cm, all fixed in Bouin's fixative. All tissues should be stained with H&E. The small intestine in addition to H&E needs to be stained with alcian blue.

additional organ preservation—brains, lungs, liver and tongues from sacrificed animals to be preserved as well. The liver tissue, the large lobe should be cut in two pieces and fixed in Bouin's fixative. The whole lungs, the tongue (dissected into two-longitudinally) and the brains are fixed in neutral buffered formalin. The remaining carcasses should be fixed in formalin.

While the invention has been described in detail with reference to certain preferred embodiments thereof, it will be understood that modifications and variations are within the spirit and scope of that which is described and claimed.

What is claimed is:

1. A pharmaceutical composition for treating cell proliferative disorders comprising a pharmaceutically acceptable carrier, a therapeutically effective amount of TGF-α, TGF-α-related polypeptide, or a functional fragment of TGF-α or TGF-α-related polypeptide.

2. The pharmaceutical composition of claim 1, further comprising a therapeutically effective amount of a chemotherapeutic agent.

3. The pharmaceutical composition according to claim 2, wherein said chemotherapeutic agent is selected from the group consisting of alkylating agents, DNA strand-breaking agents, intercalating topoisomerase II inhibitors, nonintercalating topoisomerase II inhibitors, DNA minor groove binders, antimetabolites, tubulin-binding agents that when bound to tubulin prevent formation of microtubules, hormones, asparaginase and hydroxyurea.

4. The pharmaceutical composition according to claim 2, wherein said chemotherapeutic agent is selected from the group consisting of asparaginase, hydroxyurea, cisplatin, cyclophosphamide, altretamine, bleomycin, dactinomycin, doxorubicin, etoposide, teniposide, and plicamycin.

5. The pharmaceutical composition according to claim 2, wherein said chemotherapeutic agent is selected from the group consisting of methotrexate, fluorouracil, fluorodeoxyuridine, CB3717, azacitidine, cytarabine, floxuridine, mercaptopurine, 6-thioguanine, fludarabine, pentostatin, cyctrabine, and fludarabine.

6. A method of treating a cell proliferative disorder in a mammal comprising administering to a subject in need thereof, a therapeutically effective amount of TGF-α, TGF-α-related polypeptide, or functional fragment thereof, thereby treating the disorder.

7. The method according to claim 6, wherein from about 1.0 ug/kg body weight to about 100 mg/kg body weight of TGF-α, TGF-α-related polypeptide or TGF-α or TGF-α-related polypeptide functional fragment is administered.

8. The method of claim 6, further comprising administering a therapeutically effective amount of a chemotherapeutic agent.

9. The method of claim 6, wherein from about 0.5 mg/kg body weight to about 40 mg/kg body weight of said chemotherapeutic agent is administered.

10. The method according to claim 6, wherein the TGF-α is administered orally, enterically, intravenously, peritoneally, parenterally or by injection into a tumor.

11. The method according to claim 8, wherein said chemotherapeutic agent is selected from the group consisting of alkylating agents, DNA strand-breaking agents, intercalating topoisomerase II inhibitors, nonintercalating topoisomerase II inhibitors, DNA minor groove binders, antimetabolites, tubulin-binding agents that when bound to tubulin prevent formation of microtubules, hormones, asparaginase and hydroxyurea.

12. A method according to claim 8, wherein said chemotherapeutic agent is selected from the group consisting of Asparaginase, hydroxyurea, Cisplatin, Cyclophosphamide, Altretamine, Bleomycin, Dactinomycin, Doxorubicin, Etoposide, Teniposide, and Plicamycin.

13. The method according to claim 8, wherein said chemotherapeutic agent is selected from the group consisting of Methotrexate, Fluorouracil, Fluorodeoxyuridine, CB3717, Azacitidine, Cytarabine, Floxuridine, Mercaptopurine, 6-Thioguanine, Fludarabine, Pentostatin, Cyctrabine, and Fludarabine.

14. The method of according to claim 8, wherein said chemotherapeutic agent is Uracil mustard, Chlormethine, Cyclophosphamide, Ifosfamide, Melphalan, Chlorambucil, Pipobroman, Triethylenemelamine, Triethylenethiophosphoramine, Busulfan, Carmustine, Lomustine, Streptozocin, Dacarbazine, Temozolomide, Methotrexate, 5-Fluorouracil, Floxuridine, Cytarabine, 6-Mercaptopurine, 6-Thioguanine, Fludarabine phosphate, Pentostatine, Gemcitabine, Vinblastine, Vincristine, Vindesine, Bleomycin, Dactinomycin, Daunorubicin, Doxorubicin, Epirubicin, Idarubicin, Paclitaxel, Mithramycin, Deoxycoformycin, Mitomycin-C, L-Asparaginase, Interferons, Etoposide, Teniposide 17.alpha.-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Goserelin, Cisplatin, Carboplatin, Hydroxyurea, Amsacrine, Procarbazine, Mitotane, Mitoxantrone, Levamisole, Navelbene, CPT-11, Anastrazole, Letrazole, Capecitabine, Reloxafine, Droloxafine, or Hexamethylmelamine.

15. The method of claim 6, wherein said cell proliferative disorder is lung cancer, pancreatic cancer, colon cancer, myeloid leukemia, melanoma, glioma, thyroid follicular cancer, bladder carcinoma, myelodysplastic syndrome, breast cancer, low grade astrocytoma, astrocytoma, glioblastoma, medulloblastoma, renal cancer, prostate cancer, endometrial cancer and neuroblastoma.

16. The method of claim 6, further comprising radiation therapy.

17. The method of claim 8, wherein TGF-α and said chemotherapeutic agent are administered simultaneously.

18. The method of claim 8, wherein TGF-α and said chemotherapeutic agent are administered sequentially.

19. The method of claim 8, wherein said chemotherapeutic agent is administered prior to TGF-α.

20. A method of treatment of a cell proliferative disorder in a subject in need thereof comprising introducing into cells of a host subject, an expression vector comprising a polynucleotide sequence encoding TGF-α or a biologically functional fragment thereof, in operable linkage with a promoter.

21. The method of claim 20, wherein the expression vector is introduced into the subject's cells ex vivo and the cells are then reintroduced into the subject.

22. The method of claim 20, wherein the expression vector is an RNA virus.

23. The method of claim 22, wherein the RNA virus is a retrovirus.

24. The method of claim 20, wherein the subject is a human.
